(19) United States
(12) Patent Application Publication
(10) Pub. No.: US 2026/0172675 A1
(43) Pub. Date: Jun. 18, 2026

(54) SEIZURE MONITORING

(71) Applicant: Welch Allyn, Inc., Skaneateles Falls, NY (US)

(72) Inventors: WonKyung McSweeney, Manlius, NY (US); Zhon Ye Chu, Syracuse, NY (US); Danielle R. Endres, Syracuse, NY (US); Corinn C. Fahrenkrug, Chittenango, NY (US); Michael Holtz, Rochester, NY (US); John A. Lane, Venice, FL (US); Anzhelika Polshikova, Syracuse, NY (US); David E. Quinn, Moravia, NY (US); Tyson B. Whitaker, Arden, NC (US); Gene J. Wolfe, Pittsford, NY (US)

(21) Appl. No.: 19/374,109

(22) Filed: Oct. 30, 2025

Related U.S. Application Data
(60) Provisional application No. 63/715,310, filed on Nov. 1, 2024.

(51) Int. Cl. H04N 23/667; A61B 5/00; G06V 20/52; G06V 20/70; G06V 40/20; G16H 10/20; G16H 30/20; H04N 7/18

(52) U.S. Cl. CPC H04N 23/667; A61B 5/0077; A61B 5/4094; A61B 5/4803; A61B 5/7246; A61B 5/7264; A61B 5/746; G06V 20/52; G06V 20/70; G06V 40/20; G16H 10/20; G16H 30/20; H04N 7/18; A61B 2560/0238

(57) ABSTRACT

A system for seizure monitoring controls a camera to operate under a monitoring mode to continuously record data from a subject. The system establishes one or more baselines associated with the subject while operating the camera under the monitoring mode. The system detects one or more deviations from the one or more baselines during a first time window. The one or more deviations being associated with an onset of seizure. The system adjusts one or more settings of the camera while operating the camera under the monitoring mode. When the one or more deviations from the one or more baselines persist during a second time window, the system adjusts the camera to operate under a seizure mode.

### CROSS-REFERENCE TO RELATED APPLICATION(S)

This application claims the benefit of U.S. Provisional Application No. 63/715,310, filed Nov. 1, 2024, the disclosure of which is hereby incorporated by reference in its entirety.

### BACKGROUND

Seizures are sudden, uncontrolled electrical disturbances in the brain that can cause a variety of symptoms. They can manifest as convulsions, loss of consciousness, or unusual sensations and behaviors. Seizures vary in duration and intensity, and their causes can range from epilepsy and head injuries to infections and metabolic imbalances. Diagnosis typically involves medical history, neurological exams, and tests such as an electroencephalogram (EEG). Treatment may include medications, lifestyle changes, and, in some cases, surgery. Managing seizures often involves addressing the underlying condition and preventing triggers.

When a patient experiences a seizure, there are specific symptoms before, during, and after a seizure that should be documented. However, medical professionals are not always present in the moments before a seizure occurs such that they often cannot witness the events that occur before onset of a seizure. This makes it difficult to identify the triggers of the seizure and to know how long the seizure has been occurring, which can affect health outcomes because certain interventions are recommended based on the triggers and duration of the seizure.

### SUMMARY

In general terms, the present disclosure relates to seizure monitoring. In one possible configuration, an alert is generated when one or more deviations from one or more baselines are detected, and a timer is triggered. Various aspects are described in this disclosure, which include, but are not limited to, the following aspects.

One aspect relates to a system for seizure monitoring, the system comprising: at least one processing device; and at least one memory device storing software instructions that, when executed by the at least one processing device, cause the at least one processing device to: control a camera to operate under a monitoring mode to continuously capture data from a subject; establish one or more baselines associated with the subject while operating the camera under the monitoring mode; detect one or more deviations from the one or more baselines during a first time window, the one or more deviations being associated with an onset of seizure; adjust one or more settings of the camera while operating the camera under the monitoring mode; generate an alert when the one or more deviations from the one or more baselines persist during a second time window; and adjust the camera to operate under a seizure mode.

Another aspect relates to a method of seizure monitoring, the method comprising: controlling a camera to operate under a monitoring mode to continuously capture data from a subject; establishing one or more baselines associated with the subject while operating the camera under the monitoring mode; detecting one or more deviations from the one or more baselines during a first time window, the one or more deviations being associated with an onset of seizure; adjusting one or more settings of the camera while operating the camera under the monitoring mode; generating an alert when the one or more deviations from the one or more baselines persist during a second time window; and adjusting the camera to operate under a seizure mode.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for seizure monitoring.

FIG. 2 schematically illustrates an example of the system of FIG. 1 that includes a seizure analytics system communicatively coupled via a network to one or more devices.

FIG. 3 schematically illustrates an example of a method of seizure monitoring that can be performed by the seizure analytics system of FIG. 2.

FIG. 4 schematically illustrates an example of a method of seizure analysis that can be performed by the seizure analytics system of FIG. 2.

FIG. 5 schematically illustrates an example of a seizure detection model that can be used by the seizure analytics system of FIG. 2 to detect the onset of a seizure.

FIG. 6 schematically illustrates an example of a large language model (LLM) that can be used by the seizure analytics system of FIG. 2 to perform a post-seizure assessment.

FIG. 7 illustrates an example of an electronic medical record (EMR) displayed on a display screen of a workstation device by the seizure analytics system of FIG. 2.

### DETAILED DESCRIPTION

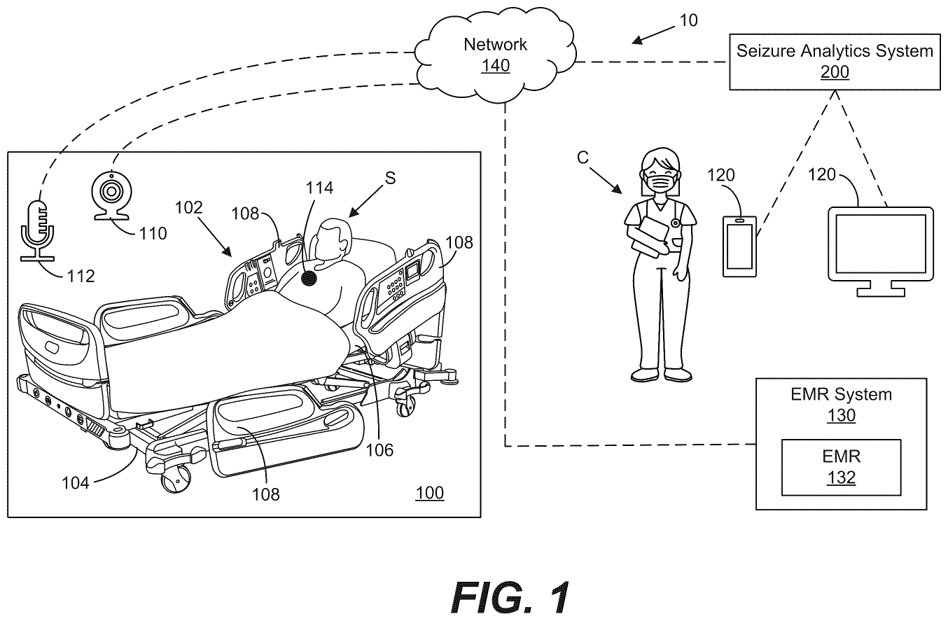

FIG. 1 schematically illustrates an example of a system 10 for seizure monitoring. As will be described in more detail, the system 10 records data before, during, and after detection of a seizure experienced by a subject S located inside an area 100. Upon detecting an onset of the seizure, the system 10 alerts caregivers C, starts a timer for recording a duration of the seizure, and scans the area 100 to ensure proper seizure precautions are followed. After the seizure ends, the system 10 annotates a recording of the data collected before, during, and after the seizure for identification of potential triggers and symptoms associated with the seizure.

The system 10 includes one or more devices that collect data from the subject S inside the area 100. The system 10 can include a support apparatus 102, one or more cameras 110, a microphone 112, and one or more sensors 114 worn by the subject S in the area 100.

Illustrative examples of the support apparatus 102 include a hospital bed, a stretcher, a wheelchair, and other types of apparatuses that can physically support the subject S inside the area 100. In the example illustrated in FIG. 1, the support apparatus 102 is a hospital bed that includes a frame 104 that supports a mattress 106. The support apparatus 102 can share aspects with the apparatus described in U.S. patent application Ser. No. 18/438,969, filed Feb. 12, 2024, entitled PATIENT SUPPORT APPARATUS HAVING VITAL SIGNS MONTORING AND ALERTING, the disclosure of which is herein incorporated by reference in its entirety.

The frame 104 can include one or more electronic motors to raise and lower the mattress 106 relative to the ground. The frame 104 can further include one or more sensors that capture measurements of physiological variables of the subject S such as heart rate, respiration rate, weight, and motion activity. For example, the one or more sensors can detect an increase in heart rate (i.e., tachycardia) and a difficulty in breathing, which are symptomatic of seizures. The one or more sensors can be positioned under the mattress 106, embedded in the mattress 106, positioned on top of the mattress 106, or can be positioned elsewhere on the frame 104.

The frame 104 further includes siderails 108 such as an upper left siderail, an upper right siderail, a lower left siderail, and a lower right siderail. The one or more electronic motors of the support apparatus 102 can be controlled to raise one or more of the siderails 108 to move from a stowed position to a deployed position, and to lower one or more of the siderails to move from the deployed position to the stowed position. When the siderails 108 are in the stowed position, the subject S is able to exit the support apparatus 102. When the siderails 108 are in the deployed position, the subject S is prevented from exiting the support apparatus 102.

The one or more cameras 110 can be configured to pan, tilt, and zoom for adjusting a view of the area 100 as well as views of the subject S, the support apparatus 102, and other objects within the area 100. The cameras 110 can include a gimbal or similar structure actuated by an electronic motor to pan the cameras 110 left and right, and to tilt the cameras 110 up and down. Also, the cameras 110 can zoom in and out by adjusting a focal length of a lens whether mechanically (e.g., mechanical zoom) or digitally (e.g., digital zoom). The one or more cameras 110 capture data of the subject S which can be analyzed to identify twitching, loss of muscle control, repeated movements, staring spells, and other movements symptomatic of a seizure.

The microphone 112 captures audio inside the area 100. The audio captured by the microphone 112 can include sounds from the subject S such as difficulty speaking, uttering nonsensical or strange words, difficulty breathing, and other sounds symptomatic of a seizure.

The one or more sensors 114 worn by the subject S in the area 100 can be used to alternatively measure the heart rate and respiration rate of the subject S. In some examples, the sensors 114 can also be used to capture additional physiological variable measurements such as to record electrical activity of the brain by recording an electroencephalogram (EEG).

The data collected by the one or more devices inside the area 100 is communicated over a network 140 to a seizure analytics system 200. As will be described in more detail, the seizure analytics system 200 analyzes the data received from the one or more devices in the area 100 to determine whether the subject S is experiencing an onset of a seizure, and if so, to generate alerts for notifying caregivers C. In some examples, the seizure analytics system 200 is integrated into a video monitoring system such as the one described in U.S. patent application Ser. No. 63/669,279, filed Jul. 10, 2024, entitled AUTOMATED PATIENT CHARTING, the disclosure of which is herein incorporated by reference in its entirety.

For example, when the seizure analytics system 200 determines that the subject S is experiencing the onset of a seizure based on the data received from the one or more devices inside the area 100, the seizure analytics system 200 generates alerts on workstation devices 120 to notify the caregivers C that the subject S requires immediate care. The alerts can include a duration of the seizure that is calculated based on a timer that is triggered based on the data received from the one or more devices inside the area 100. The alerts can further identify one or more triggers or symptoms of seizures that are detected from the data received from the one or more devices inside the area 100. The alerts generated by the seizure analytics system 200 can further identify one or more unsafe conditions inside the area 100 that should be ameliorated.

The workstation devices 120 can include portable computing devices such as tablet computers and smartphones carried by caregivers C. The workstation devices 120 may also include stationary monitors such as desktop monitors or wall mounted monitors that are located in a designated area of a healthcare facility such as a nurses'station within a hospital.

Further, the seizure analytics system 200 stores the data collected from the one or more devices before the onset of the seizure, during the seizure, and after the seizure terminates. The seizure analytics system 200 can store the data collected from the one or more devices in an electronic medical record (EMR) 132 of the subject S maintained by an EMR system 130.

The EMR 132 (alternatively termed electronic health record (EHR)) operates to manage the subject S's medical history and information. The EMR system 130 can be operated by a healthcare service provider such as a hospital or medical clinic. The seizure analytics system 200 sends seizure duration time estimates, detected seizure symptoms and potential seizure triggers, and other annotations derived from the data acquired from the one or more devices inside the area 100 to the EMR system 130 via the network 140. The EMR system 130 stores the outputs of the seizure analytics system 200 in the EMR 132 of the subject S.

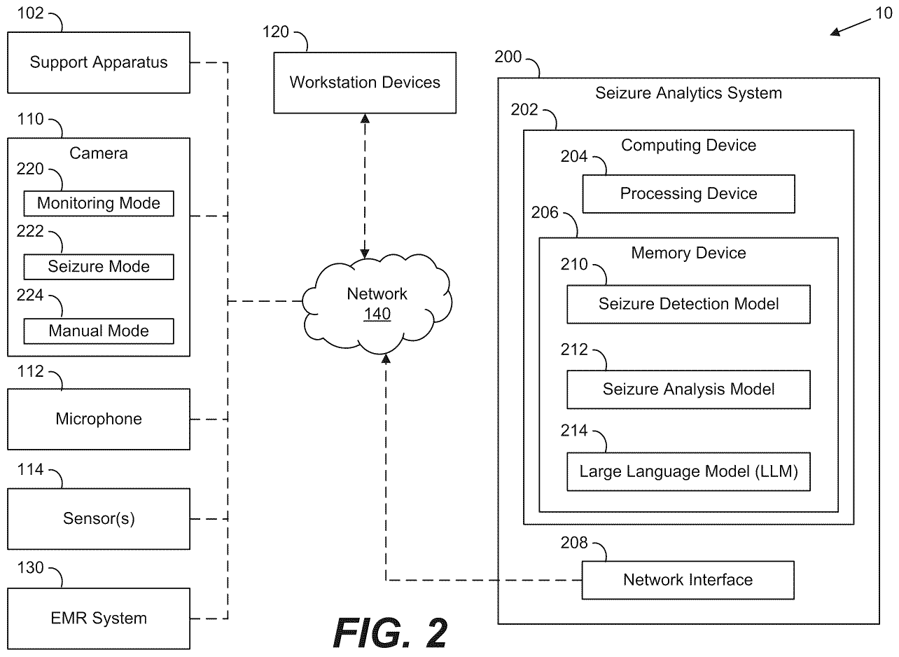

FIG. 2 schematically illustrates an example of the system 10 that includes the seizure analytics system 200 communicatively coupled via the network 140 to the one or more devices inside the area 100 including the support apparatus 102, the one or more cameras 110, the microphone 112, and the one or more sensors 114 worn by the subject S. Also, the seizure analytics system 200 is shown communicatively coupled via the network 140 to the workstation devices 120 and the EMR system 130 where the EMR 132 of the subject S is maintained.

The seizure analytics system 200 includes a computing device 202 having at least one processing device 204 and at least one memory device 206 that stores software instructions that, when executed by the at least one processing device 204, cause the at least one processing device 204 to perform the various aspects, functions, and operations described herein.

The at least one processing device 204 is an example of a processing unit such as a central processing unit (CPU). The at least one processing device 204 can include one or more CPUs. In some examples, the at least one processing device 204 includes one or more digital signal processors, field-programmable gate arrays, and/or other types of electronic circuits.

The at least one memory device 206 is an example of a computer-readable data storage device that operates to store data and instructions for execution by the at least one processing device 204. As shown in FIG. 2, the at least one memory device 206 stores a seizure detection model 210 which analyzes the data received from the one or more devices inside the area 100 to determine whether the subject S is experiencing the onset of a seizure.

The at least one memory device 206 further stores a seizure analysis model 212 that annotates the data captured by the one or more devices inside the area 100. The seizure analysis model 212 can also store the data and the annotations in the EMR 132 of the subject S.

The at least one memory device 206 further stores a large language model (LLM) 214, which is a type of artificial intelligence (AI) program that uses deep learning to generate human-like interactions to engage the subject S after the seizure ends. For example, the LLM 214 can generate questions for assessing potential triggers of the seizure and symptoms felt by the subject S, and for assessing a post-seizure condition of the subject S. When the subject S replies to the questions, the LLM 214 can adaptively generate follow-up questions.

The at least one memory device 206 includes computer-readable media, which includes any media that can be accessed by the at least one processing device 204. The computer-readable media can include computer-readable storage media and computer-readable communication media. The computer-readable storage media includes volatile and nonvolatile, removable and non-removable media implemented in any device that can store information such as computer-readable instructions, data structures, program modules, or other data. The computer-readable storage media can include random access memory, read only memory, electrically erasable programmable read only memory, flash memory, and other memory technology, including any medium that can be used to store information that can be accessed by the at least one processing device 204. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. The computer-readable communication media can include wired media such as a wired network or direct-wired connection, and wireless media such as acoustic, radio frequency, infrared, and other wireless media. Combinations of any of the above are within the scope of computer-readable media.

The seizure analytics system 200 includes a network interface 208 that allows the seizure analytics system 200 to connect to the network 140. The network interface 208 can include wired interfaces and/or wireless interfaces. For example, the network interface 208 can wirelessly connect to the network 140 such as through Wi-Fi and other wireless communications protocols. Alternatively, or additionally, the network interface 208 can connect to the network 140 using wired connections such as through Ethernet or Universal Serial Bus (USB) cables.

The network 140 can include any type of wired or wireless connections or any combinations thereof. Examples of wireless connections include Wi-Fi, Bluetooth, ultra-wideband (UWB), radio frequency identification (RFID), cellular network connections, and the like. In some examples, the network 140 is an Internet-of-things (IoT) network that connects and exchanges data between the one or more devices inside the area 100 and with other systems and devices over the Internet or other communications networks. In alternative examples, one or more of the devices inside the area 100 directly communicate with the seizure analytics system 200 without using the network 140 such as via wireless or wired direct connections.

As shown in FIG. 2, the one or more cameras 110 can each operate under a monitoring mode 220, a seizure mode 222, and a manual mode 224. The seizure analytics system 200 instructs the one or more cameras 110 to switch from the monitoring mode 220 to the seizure mode 222 when the seizure detection model 210 determines that the subject S is experiencing an onset of a seizure. The seizure analytics system 200 instructs the one or more cameras 110 to switch from the seizure mode 222 to the monitoring mode 220 when the seizure detection model 210 determines the seizure has terminated such as when the subject S is no longer exhibiting seizure symptoms based on the data captured from inside the area 100.

The one or more cameras 110 switch from the manual mode 224 to the seizure mode 222 upon receipt of an input from one or more of the workstation devices 120 that are controlled by the caregivers C. When operating under the manual mode 224, the one or more cameras 110 do not capture any data from the area 100 such that the one or more cameras 110 are turned off.

The monitoring mode 220 includes a data buffer having a default length that is purged from a memory of the one or more cameras 110 and/or from the at least one memory device 206 of the seizure analytics system 200. As an illustrative example, the data buffer can have a default length of 5 seconds, 10 seconds, 15 seconds, 30 seconds, or 60 seconds. After the default length expires, the data buffer is purged from the memory of the one or more cameras 110 and/or from the at least one memory device 206 of the seizure analytics system 200. The data buffer is purged to mitigate potential unauthorized disclosures of protected health information (PHI), which is any information in the EMR 132 that can be used to identify the subject S, and that was created, used, or disclosed in the course of providing healthcare services to the subject S.

When operating under the seizure mode 222, the one or more cameras 110 do not purge data after expiration of a data buffer. Instead, the data captured by the one or more cameras 110 is continuously recorded until the cameras revert back to the monitoring mode 220.

The one or more cameras 110 when operating under the seizure mode 222 can also have a higher frame rate than when operating under the monitoring mode 220 such that the data captured during the seizure mode 222 has a higher resolution than the data captured during the monitoring mode 220. This can reduce bandwidth consumption over the network 140 when the seizure analytics system 200 determines that the subject S is not experiencing a seizure.

The monitoring mode 220 and the seizure mode 222 may also utilize different imaging modalities. For example, the monitoring mode 220 detects infrared images and the seizure mode 222 detects visible red-green-blue (RGB) images, or conversely, the monitoring mode 220 detects visible RGB images and the seizure mode 222 detects infrared images. Thus, the one or more cameras 110 can toggle between different imaging modalities depending on their mode of operation. The one or more cameras 110 under the monitoring mode 220 and/or the seizure mode 222 can also capture RGB-Depth images, and other types of imaging modalities. Also, the monitoring mode 220 may include filters that blur or obfuscate the subject S, while the seizure mode 222 does not include filters that blur or obfuscate the subject S.

In further examples, a transceiver is positioned in the area 100 to transmit millimeter waves and to receive reflections of the millimeter waves off the subject S to detect twitching, shaking, and other micro-movements of the subject S. The transceiver can share aspects with the movement detection device described in U.S. patent application Ser. No. 17/102,683, filed Nov. 24, 2020, entitled MICRO-MOVEMENT AND GESTURE DETECTION USING RADAR, the disclosure of which is herein incorporated by reference in its entirety.

Once a seizure is detected, the seizure analytics system 200 enters the seizure mode 222 which can also include automatically adjusting the one or more cameras 110 to be in an ideal recording position. The seizure analytics system 200 can also have control of the lighting inside the area 100 and the positioning and orientation of the support apparatus 102, ensuring that each device inside the area 100 is in an optimal position for recording and monitoring the seizure event. For example, the seizure analytics system 200 can adjust the lighting inside the area 100 to ensure visibility of the subject S in the images captured by the one or more cameras 110, as well as repositioning the support apparatus 102 to provide a better angle, and switching to an appropriate imaging modality (e.g., RGB or IR) based on the lighting conditions inside the area 100 and/or the time of day. These adjustments enable the seizure analytics system 200 to capture high quality video footage of the seizure event, which can be crucial for accurate diagnosis and treatment planning by healthcare professionals, as will be described in further detail below.

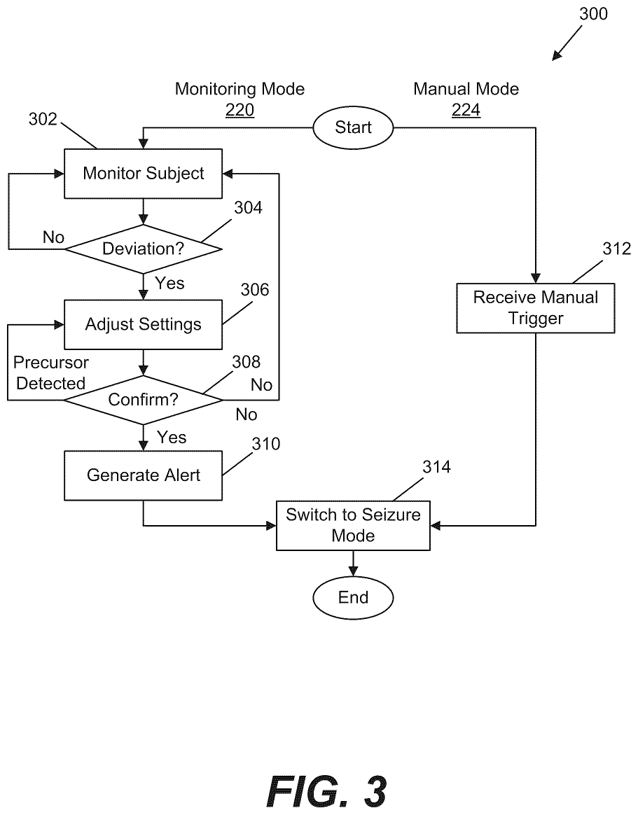

FIG. 3 schematically illustrates an example of a method 300 of seizure monitoring that can be performed by the seizure analytics system 200. In certain examples, the operations of the method 300 are performed by the seizure detection model 210 when installed on the seizure analytics system 200. As shown in FIG. 3, the method 300 can start with the monitoring mode 220 that includes controlling the one or more cameras 110 to continuously capture data from the subject S while operating under a default data buffer length. Alternatively, the method 300 can start with the manual mode 224 that includes having the one or more cameras 110 turned off.

When starting in the monitoring mode 220, the method 300 includes an operation 302 of monitoring the subject S under the monitoring mode 220. In accordance with the examples described above, operation 302 can include controlling the one or more cameras 110 to continuously capture data from the subject S while operating the one or more cameras 110 under one or more default settings such as a default data buffer length and a default frame rate.

Operation 302 can include establishing one or more baselines associated with the subject S. The baselines characterize behavior that is normal for the subject S while being monitored by the one or more devices inside the area 100. For example, the one or more baselines are established for physiological variables (e.g., heart rate and/or respiration rate), motion attributes, and/or speech characteristics. Operation 302 can include updating the one or more baselines associated with the subject S based on the continuously captured data.

Operation 302 can include establishing the physiological variables baselines based on the data captured by the one or more sensors on the frame 104 of the support apparatus 102. Operation 302 can also include establishing the physiological variables baselines based on the data captured by the one or more sensors 114 worn by the subject S. The physiological variables baselines can be established for heart rate, respiration rate, brain activity, and/or motion activity.

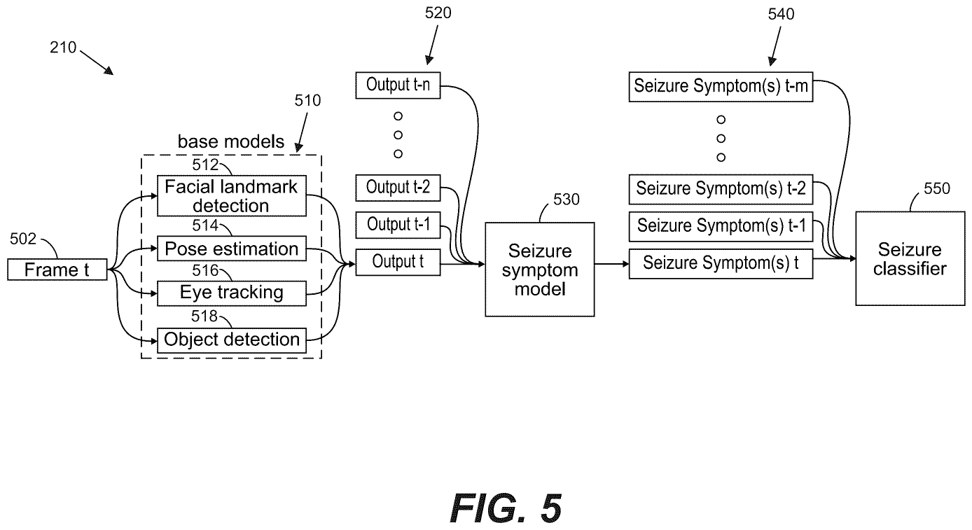

Operation 302 can include establishing the motion attribute baselines based on a pose estimation model 514 (see FIG. 5) that detects the position and orientation of the subject S in the area 100 based on the data captured by the one or more cameras 110. Operation 302 can also include using a facial landmark detection model 512 (see FIG. 5) that detects and localizes in real-time specific landmarks on the face of the subject S, such as the eyes, nose, mouth, and chin for facial expression analysis and/or head pose estimation. The pose estimation model 514 and/or the facial landmark detection model 512 can be used to determine motion attribute baselines related to shaking, twitching, and starring that are relevant to determining an onset of a seizure.

Operation 302 can include establishing the speech characteristics baselines based on a speech to text analysis model that converts the data recorded by the microphone 112 into text that can be analyzed by the seizure analytics system 200 such as to identify whether the subject S is speaking nonsensically by saying things that are unreasonable or have no meaning.

The method 300 includes an operation 304 of determining whether there are one or more deviations from the one or more baselines established in operation 302. The one or more deviations are associated with symptoms of an onset of a seizure. The one or more deviations can include deviations from the physiological variable baselines, the motion attributes baselines, and the speech characteristics baselines established in operation 302 for the subject S. The one or more deviations can include detection of a previously unseen event (e.g., twitching when the subject S has not previously exhibited switching), or a change in a frequency or a length of a seizure symptom (e.g., when the subject S is exhibiting a starring spell that is longer than staring spells previously exhibited by the subject S while being monitored in the area 100).

As an illustrative example, when operation 302 establishes a speech characteristics baseline that identifies no speech impediment for the subject S, and when the microphone 112 detects the subject S as having slurred and/or meaningless speech, such speech characteristics are considered a deviation from the speech characteristics baseline established for the subject S (i.e., “Yes” in operation 304). When operation 302 establishes a speech characteristics baseline that identifies the subject S as frequently having slurred speech, when the microphone 112 detects slurred speech from the subject S, the slurred speech is not considered to be a deviation from the speech characteristics baseline established for the subject S (i.e., “No” in operation 304).

As another illustrative example, when operation 302 establishes a motion attribute baseline that identifies the subject S as having normal fine motor skills, and when the data captured by the one or more cameras 110 detects the subject S is exhibiting uncontrolled twitching or shaking, such motion attributes are considered a deviation from the motion attribute baseline established for the subject S (i.e., “Yes” in operation 304). When operation 302 establishes a motion attribute baseline that identifies the subject S as frequently exhibiting twitching or shaking, and when the data from the one or more cameras 110 is analyzed to detect twitching or shaking exhibited by the subject S, such motion attributes are not considered to be deviations from the baseline established for the subject S (i.e., “No” in operation 304).

In some examples, operation 304 can include determining potential triggers of the seizure based on the data captured by the one or more cameras 110 while operating under the monitoring mode 220, and/or based on the data captured by the microphone 112. Potential triggers can include a flashing light in the area 100 or medication administered to the subject S.

As shown in FIG. 3, when operation 304 detects that there are no deviations from the one or more baselines established in operation 302 (i.e., “Yes” in operation 304), the method 300 can return to operation 302 to continuously monitor and updates the one or more baselines of the subject S. When operation 304 detects that there are no deviations from the one or more baselines established in operation 302 (i.e., “Yes” in operation 304), the method 300 proceeds to an operation 306 of adjusting one or more settings of the one or more cameras 110.

Operation 306 can include increasing the data buffer of the one or more cameras 110 from the default length to an extended length. The extended length of the data buffer allows additional data to be captured for analysis to confirm whether the one or more deviations from the one or more baselines are indicative of a seizure, or not. Thus, the extended length of the data buffer increases the quantity of data captured before confirmation of the seizure. In some examples, operation 306 can also include increasing the frame rate of the one or more cameras 110 such as to have a higher resolution for the data captured before confirmation of the seizure.

The method 300 includes an operation 308 of determining whether the one or more deviations detected in operation 304 are confirmed, or not. For example, operation 304 can include detecting the one or more deviations from the one or more baselines during a first time window, and when the one or more deviations are detected as persisting during a second time window, operation 308 confirms the deviations (i.e., “Yes” in operation 308). When the one or more deviations are not detected as persisting during the second time window, operation 308 does not confirm the deviations (i.e., “No” in operation 308), and the method 300 can return to operation 302 to continuously monitor and update the one or more baselines of the subject S.

Alternatively, when the one or more deviations detected in operation 304 are determined not to persist during the second time window but operation 308 determines that one or more previously undetected deviations are present during the second time window, the method 300 can return to operation 306 to adjust the settings of the one or more cameras 110 once more such as to further increase the data buffer and/or to further increase the frame rate of the one or more cameras 110 and to continue to monitor the one or more deviations from the one or more baselines for another time window to confirm whether the deviations persist or not.

When operation 308 confirms the deviations (i.e., “Yes” in operation), the method 300 proceeds to an operation 310 of generating an alert on the one or more of the workstation devices 120 for notifying the caregivers C that the subject S requires immediate care because the subject S is experiencing the onset of a seizure. As discussed above, the workstation devices 120 can include portable computing devices such as tablet computers and smartphones carried by caregivers C, and may also include stationary monitors such as desktop monitors or wall mounted monitors that are located in a designated area of a healthcare facility.

The method 300 proceeds to an operation 314 of switching the one or more cameras 110 from the monitoring mode 220 to the seizure mode 222. In some examples, operations 310, 314 occur substantially at the same time. Alternatively, the operation 314 of switching the one or more cameras 110 from the monitoring mode 220 to the seizure mode 222 can occur before the operation 310 of generating the alert on the one or more of the workstation devices 120.

The seizure mode 222 triggers a timer for recording a duration of the seizure. When a caregiver C is not present in the area 100 before the onset of a seizure occurs, the caregiver C is unable to witness the beginning of the seizure such that they are unable to determine a duration of the seizure. This is particularly significant because certain interventions are recommended based on the duration of the seizure. At least one advantage of the method 300 is that the timer is triggered regardless of whether a caregiver C is present or not in the area 100 such that the timer can be used to identify which interventions to take based on the duration of the seizure.

Further, as discussed above, the seizure mode 222 can differ from the monitoring mode 220 by operating the one or more cameras 110 to capture data without purging a data buffer after expiration of the data buffer. Also, the one or more cameras 110 can operate under a higher frame rate such that the one or more cameras 110 capture data having a higher resolution than when capturing the data while operating in the monitoring mode 220. Further, in some examples, the one or more cameras 110 when operating in the seizure mode 222 may capture the data under a different imaging modality than when operating in the monitoring mode 220.

In alternative examples where the method 300 starts with the manual mode 224 with the one or more cameras 110 turned off, the method 300 receives a manual trigger such as from a workstation device 120 operated by a caregiver C. The manual trigger can be received when the caregiver C determines that the subject S is exhibiting symptoms of a seizure. Upon receipt of the manual trigger, the method 300 proceeds to the operation 314 of switching the one or more cameras 110 from the monitoring mode 220 to the seizure mode 222. In such examples, the one or more cameras 110 are turned on to begin to operate under the seizure mode 222.

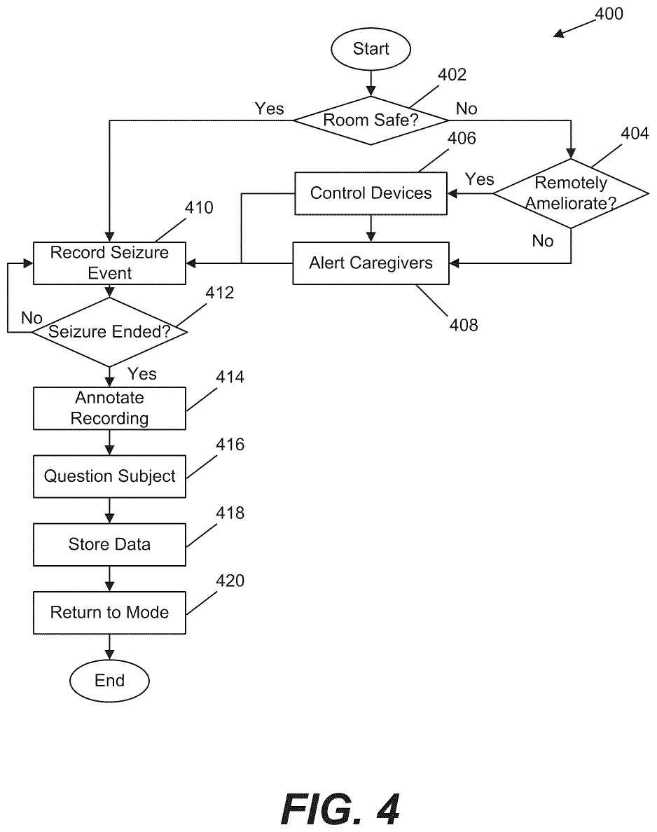

FIG. 4 schematically illustrates an example of a method 400 of seizure analysis that can be performed by the seizure analytics system 200. In certain examples, the operations of the method 400 are performed by the seizure analysis model 212. In some examples, the operations of the method 400 are performed following completion of operation 314 in the method 300 such that the one or more cameras 110 are operating under the seizure mode 222.

As shown in FIG. 4, the method 400 includes an operation 402 of determining whether there are one or more unsafe conditions around the subject S inside the area 100. In some examples the one or more unsafe conditions can be determined based on the data captured by the one or more cameras 110. An example of an unsafe condition can include one or more of the siderails 108 are in the stowed position such that the subject S can fall off the support apparatus 102 while experiencing a seizure. Another example of an unsafe condition can include the frame 104 lifting the mattress 106 high off the ground instead of in a low position closer to the ground. In other examples, the one or more unsafe conditions can be detected by other sensors inside the area 100 such as one or more sensors on the support apparatus 102, the microphone 112, and/or the one or more sensors 114 worn by the subject S.

When operation 402 determines that there is at least one unsafe condition inside the area 100 (i.e., “No” in operation 402), the method 400 proceeds to an operation 404 of determining whether the at least one unsafe condition can be remotely ameliorated. When the at least one unsafe condition can be remotely ameliorated (i.e., “Yes” in operation 404), the method 400 proceeds to an operation 406 of remotely controlling one or more devices in the area 100 to ameliorate the unsafe condition. For example, operation 406 can include controlling the one or more electronic motors to lower the frame 104 such that the mattress 106 is positioned closer to the ground. As another example, operation 406 can include controlling the one or more electronic motors to raise the siderails 108 from the stowed position to the deployed position. As another example, operation 406 can include inflating the mattress 106 to make it easier to turn the subject S on their side and/or to adjust one or more relative angles between a head section, a thigh section, and a foot section of the support apparatus 102. Operation 406 can further include adjusting an ambient lighting, temperature, or other environmental conditions in the area 100. In some examples, operation 406 includes identifying the remote control action that was performed to ameliorate the unsafe condition in the alert generated in operation 310 of the method 300.

In some examples, operation 406 includes emitting one or more phrases or words via a speaker inside the area 100 and requesting the subject S to memorize the one or more phrases or words, which if the subject S remembers after the seizure event ends, can help to diagnosis the condition of the subject S that is causing the seizures. In some examples, the one or more phrases or words can be annotated in the data that is stored to the EMR 132 of the subject S.

When the at least one unsafe condition cannot be remotely ameliorated (i.e., “No” in operation 404), the method 400 proceeds to an operation 408 of alerting the caregivers C about the one or more unsafe conditions. In some examples, operation 408 includes identifying the at least one unsafe condition in the alert generated in operation 310 of the method 300. In some examples, operation 408 includes providing a recommendation to ameliorate the at least one unsafe condition in the alert generated in operation 310 of the method 300.

As an illustrative example, operation 408 can include a recommendation to place seizure pads on the siderails 108 of the support apparatus 102 to mitigate the impact of the subject S hitting the siderails 108 due to uncontrolled bodily movements such as from tremors and shaking. Further illustrative examples of recommendations to ameliorate unsafe conditions can include recommendations to position the subject S in the lateral position, to place a pillow under the subject S's head while laying on the mattress 106 of the support apparatus 102, to place a bedside suction device to prevent the subject S from aspirating liquid during the seizure, and/or to provide privacy such as by drawing curtains or closing blinds in the area 100.

The method 400 includes an operation 410 of recording the seizure event by operating the one or more cameras 110 in the seizure mode 322. Operation 410 includes operating the one or more cameras 110 in the seizure mode 222. Operation 410 can include operating the one or more cameras 110 to pan left and right, to tilt up and down, and to zoom in and out by adjusting a focal length of a lens whether mechanically (e.g., mechanical zoom) or digitally (e.g., digital zoom) to follow the movements of the subject S inside the area 100. Operation 410 can also include recording the seizure event using the microphone 112.

The method 400 includes an operation 412 of determining whether the seizure has ended. Operation 412 can include detecting the termination of the seizure based on the data recorded by the one or more cameras 110 while operating the one or more cameras 110 under the seizure mode. In some examples, operation 412 can also include detecting termination of the seizure based on the data recorded by the microphone 112. Operation 412 can include determining whether the one or more deviations from the one or more baselines continue to persist, or not. When it is determined that the seizure has not yet terminated (i.e., “No” in operation 412), the method 400 returns to operation 410 to continue to record the seizure event.

When it is determined that the seizure has terminated (i.e., “Yes” in operation 412), the method 400 proceeds to an operation 414 of annotating the recording of the seizure. Operation 414 can be performed by the seizure analysis model 212.

Operation 414 can include annotating the data captured by the one or more cameras 110 while operating under the seizure mode 322 to identify symptoms associated with the seizure such as twitching, loss of muscle control, repeated movements, racing heart (i.e., tachycardia), trouble breathing, staring spell, and other symptoms. Operation 414 can include annotating the data captured by the microphone 112 to identify symptoms associated with the seizure such as difficulty speaking, saying strange words, and trouble breathing. Operation 414 can also include annotating the data captured by the one or more cameras 110 and/or the microphone 112 to identifying potential triggers of the seizure based on the data captured by the one or more cameras 110 while operating in the monitoring mode 220 and/or by the microphone 112.

In some examples, the method 400 includes an operation 416 of questioning the subject S for assessing symptoms felt by the subject S before and during the seizure, and for assessing a post-seizure condition of the subject S. In some examples, the series of questions is dynamically updated based on the responses provided by the subject S. In some examples, operation 416 includes using the LLM 214 to generate the series of questions. In some examples, operation 416 includes improving the seizure detection model 210 based on answers received from the subject S in response to the questions provided by the LLM 214. The answers received from the subject S can be recorded by the one or more cameras 110 and/or the microphone 112.

In some examples, the method 400 includes an operation 418 of storing the data captured by the one or more cameras 110 and/or the microphone 112 to the EMR 132 associated with the subject S. Operation 418 can also include storing the annotations generated in operation 414 to the EMR 132 associated with the subject S. Operation 418 can also include storing the answers to the series of questions received in operation 416 to the EMR 132.

Once enough time has passed that the subject S is no longer experiencing seizure symptoms, the method 400 proceeds to an operation 420 of returning the operation of the one or more cameras 110 and/or the microphone 112 from the seizure mode 222 back to the monitoring mode 220. Alternatively, operation 420 can include returning the seizure mode 222 back to the manual mode 224 where the one or more cameras 110 and/or the microphone 112 are turned off.

In view of the foregoing, the methods 300, 400 when performed together record data on the subject S before, during, and after the seizure. Further, certain operations of the methods 300, 400 are automatically triggered such as when operating under the monitoring mode 220. Alternatively, certain operations of the methods 300, 400 are manually triggered such as when operating under the manual mode 224 that starts with the one or more cameras 110 turned off.

In the case of the monitoring mode 220, the one or more cameras 110 are turned on for continuously monitoring the subject S. Before the seizure occurs, the one or more cameras 110 and/or the microphone 112 monitor for symptoms of an upcoming or ongoing seizure such as difficulty speaking, saying strange words, twitching, loss of muscle control, repeated movements, racing heart (i.e., tachycardia), trouble breathing, and staring spell. The symptoms of an upcoming or ongoing seizure can be detected through one or more combinations of a pose estimation model, a facial land-marking model, and/or a speech to text analysis model. In this manner, a complete data set is acquired by performance of the methods 300, 400 to provide better diagnoses of the subject S and improve the healthcare provided to the subject S.

FIG. 5 schematically illustrates an example of the seizure detection model 210 that can be used by the seizure analytics system 200 to detect the onset of a seizure based on the data captured by the one or more cameras 110 in the area 100. In the illustrative example shown in FIG. 5, for a given frame 502 (i.e., an image at time t) captured by a camera 110, the seizure detection model 210 can utilize one or more combinations of base models 510 such as a facial landmark detection model 512, a pose estimation model 514, an eye tracking model 516, an object detection model 518, and other models for analysis of the frame 502.

The facial landmark detection model 512 detects and localizes in real-time specific landmarks on the face of the subject S, such as the eyes, nose, mouth, and chin for facial expression analysis and/or head pose estimation. The pose estimation model 514 detects the position and orientation of the subject S in the area 100. The eye tracking model 516 can track the eye gaze and eye movement of the subject S over time. The object detection model 518 can detect objects inside the area 100 such as hazards that can result in an unsafe condition.

The base models 510 produce an output 520 (i.e., an output at time t) for analysis by a seizure symptom model 530. In some examples, the output 520 is a vector output that include a plurality of values calculated by the base models 510. The seizure symptom model 530 can include a combination of models that uses subsets of the output 520 (i.e., an output at time t) over predefined time window n to detect individual symptoms of a seizure such as by calculating scores for each symptom of a seizure. The seizure symptom model 530 can use all outputs 520 from the base models 510 within the time window n to determine a presence or an absence of certain symptoms associated with seizures. Alternatively, the seizure symptom model 530 can sample down to utilize a subset of the outputs 520 from the base models 510 to determine the presence or the absence of other types of symptoms associated with seizures.

The seizure symptom model 530 produces one or more seizure symptoms 540 at time t. The one or more seizure symptoms 540 detected at time t can include twitching, loss of muscle control, repeated movements, a staring spell, and other types of movements and motions.

As further shown in FIG. 5, the one or more seizure symptoms 540 detected at time t are fed into a seizure classifier model 550 that can be used determine whether an onset of a seizure is likely based on the one or more seizure symptoms 540. For example, the seizure classifier model 550 uses the one or more seizure symptoms 540 over a predefined time window m to determine whether an active seizure is detected at time t. The size of the predefined time window m can dynamically react to the outputs of the seizure symptom model 530 at time t. For example, when the seizure symptom model 530 detects an increase in symptom severity or frequency, the size of the predefined time window m can be increased to increase the amount of information inputted into the seizure classifier model 550 to enhance its accuracy.

The seizure classifier model 550 is tuned based on the one or more baselines established for the subject S. For example, the seizure classifier model 550 can ignore some of the seizure symptoms 540 when these symptoms are typically exhibited by the subject S.

In some examples, the seizure classifier model 550 generates a confidence level that quantifies a likelihood or probability that the seizure event is true. In some further examples, the seizure classifier model 550 classifies the seizure based on the one or more seizure symptoms 540. For example, the seizure classifier model 550 can determine whether the subject S is exhibiting a focal onset aware seizure, a focal impaired awareness seizure, a generalized motor seizure, a generalized nonmotor (absence) seizure, a tonic seizure, an atonic seizure, a myoclonic seizure, a clonic seizure, and other types of seizure classifications.

The one or more seizure symptoms 540 that are fed into the seizure classifier model 550 can include staring which occurs when the subject S stops what they are doing and just stares into space without responding to anything around them. In the data captured by the one or more cameras 110, the subject S will appear as frozen for a few seconds or minutes. The subject S may have their eyes open when staring. This kind of seizure is often called an absence seizure, and it can happen so quickly that the caregivers C may not even notice it. The detection of staring by the subject S can be accomplished via a combination of the base models 510 such as the facial landmark detection model 512, the pose estimation model 514, and the eye tracking model 516. Each of the base models 510 can generate an output 520 that is fed into the seizure symptom model 530, and the seizure symptom model 530 uses the outputs 520 of the base models 510 to determine whether staring is detected as a seizure symptom 540.

The one or more seizure symptoms 540 fed into the seizure classifier model 550 can further include limb twitching which occurs when the arms or legs of the subject S start to twitch, jerk, or shake suddenly in an uncontrolled manner due to muscles in the arms or legs being turning on and off at a fast rate. Twitching can be detected based on the outputs 520 of the pose estimation model 514 where the data captured by the one or more cameras 110 has a high enough frame rate to capture sudden fast movements. The outputs 520 from the pose estimation model 514 can include tracking the locations of limbs and joints of interest (e.g., arms, legs, wrists, elbows, shoulders, and the like) over a period of time to determine whether there are sudden abnormal changes in their location within a range of a speed or pattern indicative of twitching. The outputs 520 from the pose estimation model 514 can be fed into the seizure symptom model 530 to determine whether limb twitching is detected as a seizure symptom 540.

The one or more seizure symptoms 540 fed into the seizure classifier model 550 can include stiff muscles which occur when the muscles of the subject S tighten and the subject S is unable to relax them. For example, when the subject S has a seizure, their body may tense up all over or just in one area, and the subject S cannot control it as if their muscles are being told to squeeze really hard without the subject S wanting to do it. This symptom also be detected via outputs 520 from the pose estimation model 514. The outputs 520 can then be classified between tense versus relaxed state, and the duration of such states is detected by the seizure symptom model 530 to determine whether stiff muscles are detected as a seizure symptom 540.

The one or more seizure symptoms 540 fed into the seizure classifier model 550 can further include difficulty breathing which can happen when the seizure causes the muscles in the chest to tighten up, making it hard for the subject S to take in air. The subject S may even stop breathing for a short time during a seizure. The difficulty breathing may also be accompanied with racing heartbeat (i.e., tachycardia). The difficulty breathing and/or racing heartbeat of the subject S can be monitored via the one or more sensors on the support apparatus 102, the one or more cameras 110, the microphone 112, and/or the one or more sensors 114 worn by the subject S. In some examples, movement and edge tracking techniques described in U.S. patent application Ser. No. 18/588,771, filed Feb. 27, 2024, entitled RESPIRATION MONITORING, the disclosure of which is herein incorporated by reference in its entirety, can be used to detect difficulty breathing and/or racing heartbeat. In some examples, outputs 520 from the pose estimation model 514 can be fed into the seizure symptom model 530 to determine whether difficulty breathing and/or racing heartbeat is detected as a seizure symptom 540.

The seizure symptoms 540 fed into the seizure classifier model 550 can include repeated movements by the subject S such as blinking, lip-smacking, and hand movements (e.g., picking, button pushing, etc.). These seizure symptoms 540 can be detected via outputs 520 from the facial landmark detection model 512 and/or the pose estimation model 514 that are fed into the seizure symptom model 530 to detect repetitiveness that is outside of the baseline normal behavior of the subject S. These seizure symptoms 540 can then be fed into the seizure classifier model 550 that determines whether the subject S is likely experiencing onset of a seizure.

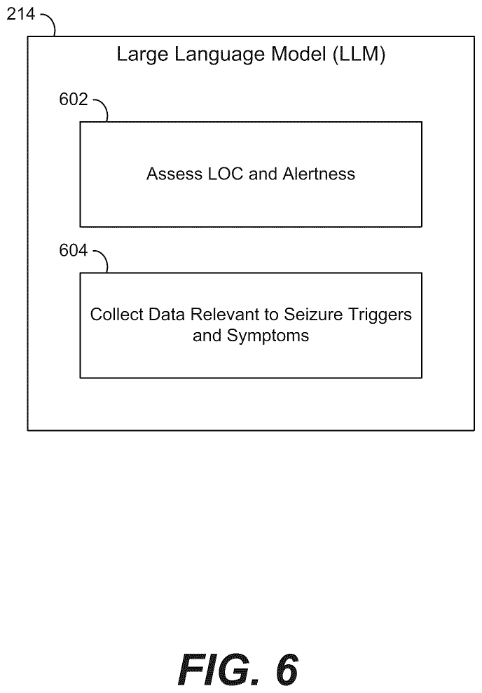

FIG. 6 schematically illustrates an example of the large language model (LLM) 214 that can be used by the seizure analytics system 200 to perform a post-seizure assessment. As described above, once the seizure analytics system 200 has detected that the seizure event has ended based on the data collected from the one or more devices inside the area 100, the seizure analytics system 200 can utilize the LLM 214 to engage the subject S with a series of questions to assess their post-seizure condition and gather information about the seizure event. For example, the LLM 214 can be used during operation 416 of the method 400 of FIG. 4.

The LLM 214 dynamically generates the questions to serve multiple purposes. For example, the questions can be organized into a first category 602 to assess a level of consciousness (LOC) and alertness of the subject S following termination of the seizure event. Under the first category 602, the LLM 214 can ask simple orientation questions such as: Can you tell me your name?; What is today's date?; and Do you know where you are right now?

The questions generated by the LLM 214 can be organized into a second category 604 to gather information about the onset and symptoms of the seizure. Under the second category 604, the LLM 214 can ask questions such as: Did you experience any warning signs before the seizure started?; Can you describe any sensations or feelings you had before the seizure?; Did you have a headache before or after the seizure?; Do you remember what you were doing right before the seizure started?; and What time of day did the seizure occur?

All responses from the subject S to the questions generated by the LLM 214 are recorded by the microphone 112 inside the area 100 and are processed by the LLM 214 for storage in the EMR 132 of the subject S for later review by healthcare professionals including the caregivers C. This data can help clinicians understand the subject S's seizure patterns, triggers, and post-seizure recovery, allowing for more personalized and effective treatment plans.

Further, the responses from the subject S to the questions generated by the LLM 214 can be used to improve the seizure detection model 210. For example, by incorporating the subject S's responses, the seizure analytics system 200 can continuously refine the seizure classifier model 550. As an example, when the subject S consistently reports a specific symptom before their seizures, the seizure analytics system 200 can adjust the seizure classifier model 550 to be more sensitive to that symptom. Over time, the seizure analytics system 200 can learn the seizure patterns, symptoms, and triggers of the subject S, thereby improving the ability of the seizure analytics system 200 to predict and respond to the subject S's future seizures.

Further, the seizure analytics system 200 can also adjust the questioning strategy of the LLM 214 based on the responses received from the subject S. Thus, the LLM 214 can tailor the questioning approach based on the subject S's unique needs and experiences.

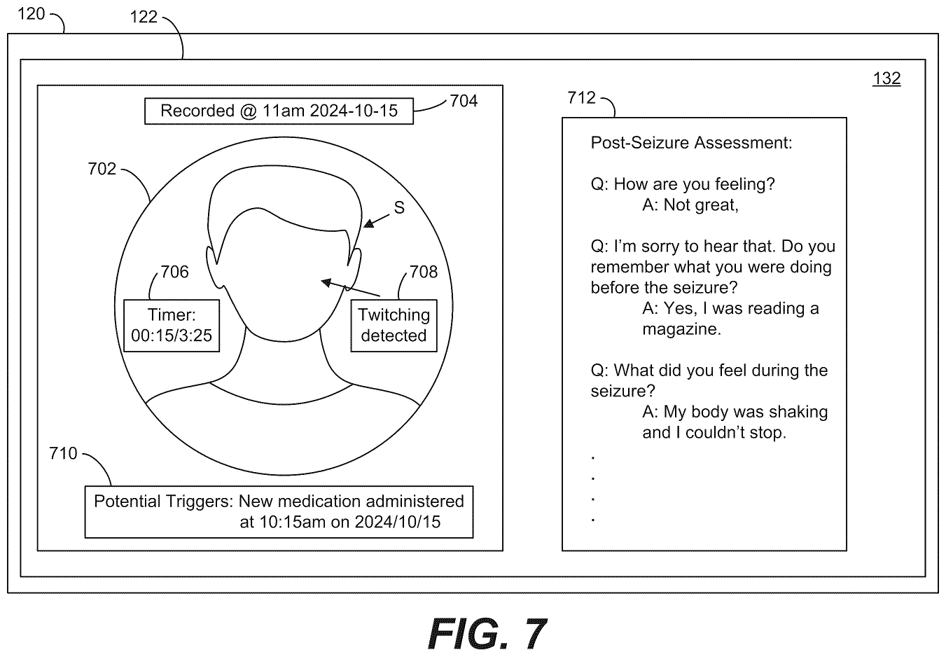

FIG. 7 illustrates an example of the EMR 132 of the subject S displayed on a display screen 122 of a workstation device 120 by the seizure analytics system 200. In this example, the EMR 132 includes a video recording 702 of the subject S captured by at least one of the cameras 110 inside the area 100. The video recording 702 is annotated by the seizure analysis model 212 to include information such as a label 704 identifying a date and time of the video recording 702. The label 704 can include additional information such as the name, date of birth, and other data identifying the subject S and/or data identifying the location of area 100.

The video recording 702 is further annotated to include a timer 706 showing a duration of the seizure. In the illustrative example shown in FIG. 7, the timer 706 shows that the video recording 702 is presently 15 seconds into a seizure event that lasts for 3 minutes and 25 seconds. The beginning and the end of the seizure event as set by the timer 706 are determined in accordance with the methods described above.

The video recording 702 is further annotated to include a label 708 identifying twitching on the face of the subject S during the seizure event. Additional labels can be added to the video recording 702 to show additional symptoms exhibited by the subject S during the seizure event. Also, the video recording 702 can be annotated to include a label 710 identifying potential triggers of the seizure event and a time stamp of the potential triggers (e.g., “New medication administered at 10:15am on 2024/10/15). It is contemplated that the seizure analysis model 212 can generate additional annotations for identifying additional types of information relevant to the video recording 702 of the seizure event for clinical assessment.

As further shown in FIG. 7, the EMR 132 can further include a transcription 712 of the post-seizure assessment performed by the LLM 214. The transcription 712 can include a summary of the questions asked by the LLM 214 and the responses from the subject S recorded by the microphone 112. In the example shown in FIG. 7, the transcription includes the response from the subject S to the question “Do you remember what you were doing before the seizure?” and to the question “What did you feel during the seizure?”. The transcription 712 is provided by way of illustrative example and it is contemplated that additional questions generated by the LLM 214 and additional answers that are recorded by the microphone 112 are possible.

The various embodiments described above are provided by way of illustration only and should not be construed to be limiting in any way. Various modifications can be made to the embodiments described above without departing from the true spirit and scope of the disclosure.

What is claimed is:

1. A system for seizure monitoring, the system comprising:
at least one processing device; and
at least one memory device storing software instructions that, when executed by the at least one processing device, cause the at least one processing device to:
control a camera to operate under a monitoring mode to continuously capture data from a subject;
establish one or more baselines associated with the subject while operating the camera under the monitoring mode;
detect one or more deviations from the one or more baselines during a first time window, the one or more deviations being associated with an onset of seizure;
adjust one or more settings of the camera while operating the camera under the monitoring mode;
generate an alert when the one or more deviations from the one or more baselines persist during a second time window; and
adjust the camera to operate under a seizure mode.

2. The system of claim 1, wherein adjust one or more settings of the camera includes adjusting at least one of a data buffer and a frame rate of the camera.

3. The system of claim 1, wherein the seizure mode triggers a timer for recording a duration of a seizure.

4. The system of claim 1, wherein the instructions, when executed by the at least one processing device, further cause the at least one processing device to:
annotate the data captured by the camera while operating under the seizure mode to identify symptoms associated with the seizure.

5. The system of claim 1, wherein the instructions, when executed by the at least one processing device, further cause the at least one processing device to:
identify potential triggers of the seizure based on the data captured by the camera while operating under the monitoring mode; and
annotate the data captured by the camera while operating under the seizure mode to include identification of the potential triggers.

6. The system of claim 1, wherein the instructions, when executed by the at least one processing device, further cause the at least one processing device to:
determine whether there are one or more unsafe conditions around the subject; and
identify the one or more unsafe conditions in the alert.

7. The system of claim 6, wherein the instructions, when executed by the at least one processing device, further cause the at least one processing device to:
remotely control one or more devices to ameliorate the unsafe conditions.

8. The system of claim 1, wherein the instructions, when executed by the at least one processing device, further cause the at least one processing device to:
utilize a large language model to engage the subject with questions for assessing symptoms felt by the subject during the seizure and a post-seizure condition of the subject.

9. The system of claim 8, wherein the instructions, when executed by the at least one processing device, further cause the at least one processing device to:
improve a seizure detection model based on answers received from the subject in response to the questions provided by the large language model.

10. The system of claim 1, wherein the instructions, when executed by the at least one processing device, further cause the at least one processing device to:
store the data recorded by the camera while operating under the seizure mode to an electronic medical record.

11. The system of claim 1, further comprising:
the camera; and
a microphone for detecting audio of the subject; and
wherein the one or more deviations from the one or more baselines are based on at least one of the data captured by the camera and the audio detected by the microphone.

12. A method of seizure monitoring, the method comprising:
controlling a camera to operate under a monitoring mode to continuously capture data from a subject;
establishing one or more baselines associated with the subject while operating the camera under the monitoring mode;
detecting one or more deviations from the one or more baselines during a first time window, the one or more deviations being associated with an onset of seizure;
adjusting one or more settings of the camera while operating the camera under the monitoring mode;
generating an alert when the one or more deviations from the one or more baselines persist during a second time window; and
adjusting the camera to operate under a seizure mode.

13. The method of claim 12, wherein adjusting the one or more settings of the camera includes adjusting at least one of a data buffer and a frame rate of the camera.

14. The method of claim 12, wherein the seizure mode triggers a timer for recording a duration of a seizure.

15. The method of claim 12, further comprising:
annotating the data captured by the camera while operating under the seizure mode to identify symptoms associated with the seizure.

16. The method of claim 12, further comprising:
identifying potential triggers of the seizure based on the data captured by the camera while operating under the monitoring mode; and
annotating the data captured by the camera while operating under the seizure mode to include identification of the potential triggers.

17. The method of claim 12, further comprising:
determining whether there are one or more unsafe conditions around the subject; and
identifying the one or more unsafe conditions in the alert.

18. The method of claim 17, further comprising:
remotely controlling one or more devices to ameliorate the unsafe conditions.

19. The method of claim 12, further comprising:
utilizing a large language model to engage the subject with questions for assessing symptoms felt by the subject during the seizure and a post-seizure condition of the subject; and
improving a seizure detection model based on answers received from the subject in response to the questions provided by the large language model.

20. The method of claim 12, further comprising:
storing the data recorded by the camera while operating under the seizure mode to an electronic medical record.
